(19) United States
(12) Patent Application Publication
(10) Pub. No.: US 2022/0047922 A1
(43) Pub. Date: Feb. 17, 2022

(54) BALANCE TRAINING SYSTEM, METHOD OF CONTROLLING THE SAME, AND CONTROL PROGRAM

(71) Applicant: TOYOTA JIDOSHA KABUSHIKI KAISHA, Toyota-shi (JP)

(72) Inventor: Kazuhiro SHINTANI, Toyokawa-shi (JP)

(73) Assignee: TOYOTA JIDOSHA KABUSHIKI KAISHA, Toyota-shi (JP)

(21) Appl. No.: 17/378,269

(22) Filed: Jul. 16, 2021

(30) Foreign Application Priority Data
Aug. 11, 2020 (JP) 2020-135601

(51) Int. Cl. A63B 26/00; A61H 1/00; A63B 24/00

(52) U.S. Cl. CPC A63B 26/003; A61H 1/005; A63B 2024/0068; A63B 2230/625; A61H 2230/625; A63B 24/0062

(57) ABSTRACT

A balance training system includes: a load detection part including a mounting surface for supporting soles of feet of a trainee in a standing state, the load detection part detecting a load received from the trainee riding on the mounting surface; a mobile body to which the load detection part is attached; a control unit that calculates a position of a center of gravity of the trainee based on the load detected by the load detection part and controls a movement of the mobile body based on a change of the position of his/her center of gravity; and a vibrator provided so as to be able to apply a vibration to the trainee. The control unit vibrates the vibrator so as to prompt the trainee to move the position of his/her center of gravity from a current position of the center of gravity to an expected position thereof.

### CROSS REFERENCE TO RELATED APPLICATIONS

This application is based upon and claims the benefit of priority from Japanese patent application No. 2020-135601, filed on Aug. 11, 2020, the disclosure of which is incorporated herein in its entirety by reference.

### BACKGROUND

The present disclosure relates to a balance training system, a method of controlling the same, and a control program.

The rehabilitation support device disclosed in Japanese Patent No. 6260811 includes a force plate on which a subject can stand, a load detection sensor for detecting a load of the subject applied to the force plate, center of gravity position detection means for detecting a center of gravity position of the subject from the load detected by the load detection sensor, and driving means. Here, the driving means moves the force plate in accordance with the moving direction of the center of gravity of the subject. cl SUMMARY

The related art has a problem that when a subject (a trainee) who needs support has difficulty in receiving voice support from an assistant due to hearing loss, higher brain disorder, or the like, the subject cannot perform effective balance training.

The present disclosure has been made in view of the above circumstances and an object thereof is to provide a balance training system, a method of controlling the same, and a control program that enable a trainee to perform effective training even when he/she has difficulty in receiving voice support from an assistant.

A first exemplary aspect is a balance training system including: a load detection part including a mounting surface for supporting soles of feet of a trainee in a standing state, the load detection part being configured to detect a load received from the trainee riding on the mounting surface; a mobile body, the load detection part being attached to the mobile body; a control unit configured to calculate a position of a center of gravity of the trainee based on the load detected by the load detection part and control a movement of the mobile body based on a change of the position of the center of gravity of the trainee; and a vibrator that is provided so as to be able to apply a vibration to the trainee, in which the control unit is configured to vibrate the vibrator so as to prompt the trainee to move the position of the center of gravity of the trainee from a current position of the center of gravity to an expected position thereof. This balance training system enables a trainee to receive support by a vibration of the vibrator even when he/she has difficulty in receiving voice support from an assistant due to hearing loss, higher brain disorder, or the like. Therefore, it is possible for the trainee to perform effective balance training.

The control unit may be configured to increase the vibration of the vibrator as a difference between the current position of the center of gravity and the expected position thereof becomes larger. This configuration enables the trainee to know how much he/she should move his/her center of gravity based on the magnitude of the vibration of the vibrator.

The control unit may be configured to apply the vibration of the vibrator to a body part of the trainee according to a direction from the current position of the center of gravity to the expected position thereof. More specifically, the control unit may be configured to apply the vibration of the vibrator to one of a toe and a heel of the foot of the trainee determined in accordance with the direction from the current position of the center of gravity to the expected position thereof. The above configuration enables the trainee to know in which direction he/she should move his/her center of gravity based on the body part to which the vibration of the vibrator has been applied.

The vibrator is provided under the load detection part. Thus, the load detection part can prevent an erroneous detection due to the load of the vibrator. Alternatively, the vibrator is attached to a predetermined body part of the trainee. Thus, the trainee can receive the vibration of the vibrator more properly.

Another exemplary aspect is a method of controlling a balance training system, the method including: detecting, by using a load detection part including a mounting surface for supporting soles of feet of a trainee in a standing state, a load received from the trainee riding on the mounting surface; and calculating a position of a center of gravity of the trainee based on the load detected by the load detection part and controlling a movement of a mobile body, to which the load detection part is attached, based on a change of the position of the center of gravity of the trainee, in which in the controlling of the movement of the mobile body, a vibrator is vibrated so as to prompt the trainee to move the position of the center of gravity of the trainee from a current position of the center of gravity to an expected position thereof. This balance training method enables a trainee to receive support by a vibration of the vibrator even when he/she has difficulty in receiving voice support from an assistant due to hearing loss, higher brain disorder, or the like. Therefore, it is possible for the trainee to perform effective balance training.

Another exemplary aspect is a control program for causing a computer to: detect, by using a load detection part including a mounting surface for supporting soles of feet of a trainee in a standing state, a load received from the trainee riding on the mounting surface; and calculate a position of a center of gravity of the trainee based on the load detected by the load detection part and control a movement of a mobile body, to which the load detection part is attached, based on a change of the position of the center of gravity of the trainee, in which in the controlling of the movement of the mobile body, a vibrator is vibrated so as to prompt the trainee to move the position of the center of gravity of the trainee from a current position of the center of gravity to an expected position thereof. This control program enables a trainee to receive support by a vibration of the vibrator even when he/she has difficulty in receiving voice support from an assistant due to hearing loss, higher brain disorder, or the like. Therefore, it is possible for the trainee to perform effective balance training.

According to the present disclosure, it is possible to provide a balance training system, a method of controlling the same, and a control program that enable a trainee to perform effective balance training even when he/she has difficulty in receiving voice support from an assistant.

The above and other objects, features and advantages of the present disclosure will become more fully understood from the detailed description given hereinbelow and the accompanying drawings which are given by way of illustration only, and thus are not to be considered as limiting the present disclosure.

### BRIEF DESCRIPTION OF DRAWINGS

FIG. 1 is a schematic perspective view of a balance training system according to a first embodiment;

FIG. 2 is a schematic side view of a part of the balance training system shown in FIG. 1;

FIG. 3 is a diagram for explaining an operation of the balance training system shown in FIG. 1;

FIG. 4 is a diagram for explaining an operation of the balance training system shown in FIG. 1;

FIG. 5 is a schematic side view showing a first modified example of the balance training system shown in FIG. 1;

FIG. 6 is a diagram for explaining an operation of the balance training system shown in FIG. 5;

FIG. 7 is a diagram for explaining an operation of the balance training system shown in FIG. 5;

FIG. 8 is a schematic side view showing a second modified example of the balance training system shown in FIG. 1;

FIG. 9 is a schematic perspective view of a balance training system according to a second embodiment;

FIG. 10 is a schematic side view of a part of the balance training system shown in FIG. 9; and

FIG. 11 is a schematic side view showing a modified example of the balance training system shown in FIG. 9.

### DESCRIPTION OF EMBODIMENTS

Hereinafter, the present disclosure will be explained through embodiments of the present disclosure. However, they are not intended to limit the scope of the present disclosure according to the claims. Further, all of the components/structures described in the embodiments are not necessarily indispensable as means for solving the problem. For clarifying the explanation, the following description and the drawings are partially omitted and simplified as appropriate. The same symbols are assigned to the same elements throughout the drawings and repeated explanations are omitted as appropriate.

### First Embodiment

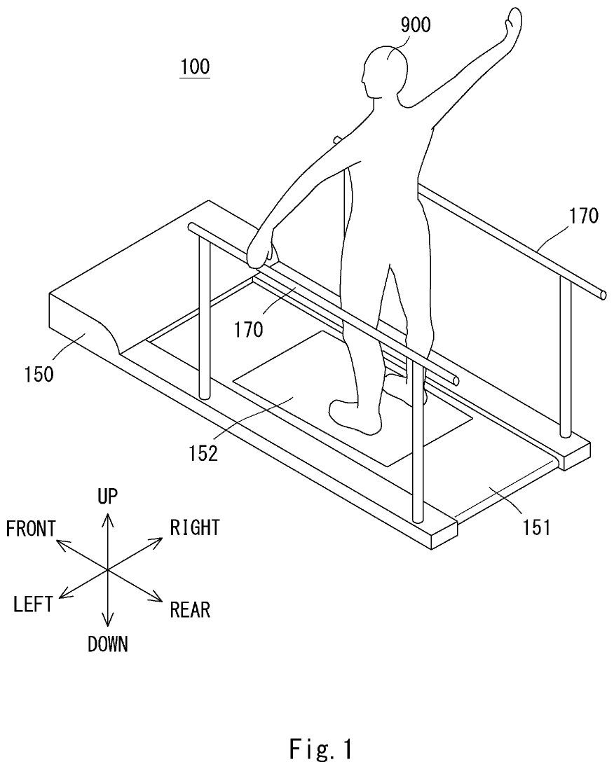

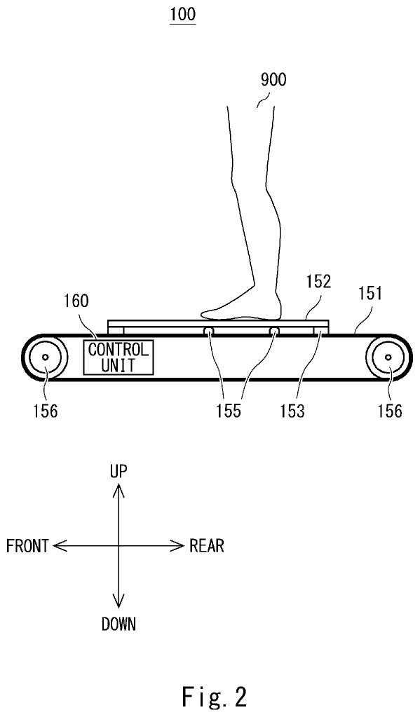

FIG. 1 is a schematic perspective view (viewed from diagonally backward to the left) of a balance training system 100 according to a first embodiment. FIG. 2 is a schematic side view (viewed from the left) of a part of the balance training system 100. The balance training system 100 may also be referred to as a balance training apparatus.

The balance training system 100 is a system for a trainee with a disability such as hemiplegia to learn to move his/her center of gravity, which the learning of moving is necessary for walking, or for a trainee with a disability in his/her ankle joint to recover the ankle joint function. For example, when a trainee 900 who wants to recover the ankle joint function tries to continue to stay riding on the balance training system 100 while maintaining his/her balance, the balance training system 100 can apply a load that can be expected to have a rehabilitation effect to the trainee 900's ankle joint.

Specifically, the balance training system 100 includes a treadmill 150, a riding plate 152, a load sensor 153, a vibrator 155, a control unit 160, and a handrail 170. The riding plate 152 and the load sensor 153 comprise the load detection part. Note that, in the following description, the up-down direction, the right-left direction, and the front-rear direction are directions based on the orientation of the trainee 900.

The treadmill 150 includes at least a ring-shaped belt (mobile body) 151, a pulley 156, and a motor (not shown). Further, the load detection part composed of the load sensor 153 and the riding plate 152 and the vibrator 155 are mounted on the belt 151.

The riding plate 152 is a riding part on which the trainee 900 rides, and has a mounting surface for supporting the sole of the trainee 900 in a standing state. As the riding plate 152, a rectangular flat plate made of, for example, polycarbonate resin, which has relatively high rigidity to withstand the riding of the trainee 900, is used. The riding plate 152 is supported on an upper surface of the belt 151 with the load sensors 153 arranged at four corners interposed therebetween.

The load sensor 153 is, for example, a load cell, and detects a load received from the foot of the trainee 900 standing on the riding plate 152. The load sensors 153 are arranged at four corners of the riding plate 152 and support the riding plate 152.

The handrail 170 is provided so as to be positioned, for example, on the side of the trainee 900 so that it can be grasped when he/she is about to lose his/her balance or when he/she feels uneasy.

The control unit 160 calculates a position of the center of gravity of the trainee 900 from the load detected by the load sensor 153 and rotates the pulley 156 based on a movement vector (a moving direction and an amount of movement) of the calculated position of the center of gravity, thereby rotating the ring-shaped belt 151. The trainee 900 standing on the belt 151 also moves with the rotation (movement) of the belt 151.

The control unit 160 also controls a vibration of the vibrator 155. The vibrator 155 is attached to the riding plate 152, for example, at a predetermined position under the riding plate 152 so that a vibration is transmitted to the sole of the foot of the trainee 900 standing on the riding plate 152. As the vibrator 155 is provided under the riding plate 152, the load detection part can prevent an erroneous detection due to the load of the vibrator 155, unlike the case in which the vibrator 155 is mounted on the riding plate 152.

The control unit 160 vibrates the vibrator 155 so as to prompt the trainee 900 to move the position of his/her center of gravity from the current position of his/her center of gravity to the expected position thereof (i.e., based on the movement vector from the current position of the center of gravity to the expected position thereof). By doing so, the trainee 900 can perform training in which his/her center of gravity is moved to the expected position thereof while receiving support from the sole of the foot by the vibration of the vibrator 155.

As described above, the balance training system 100 enables the trainee 900 to receive support by the vibration of the vibrator 155 even when he/she has difficulty in receiving voice support from an assistant due to hearing loss, higher brain disorder, or the like. Therefore, it is possible for the trainee 900 to perform effective balance training.

### (Operation of Balance Training System 100)

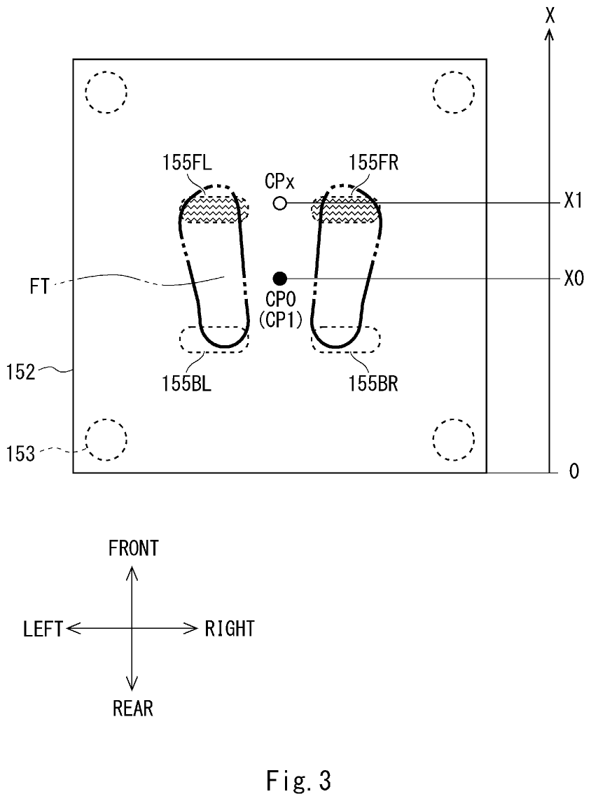

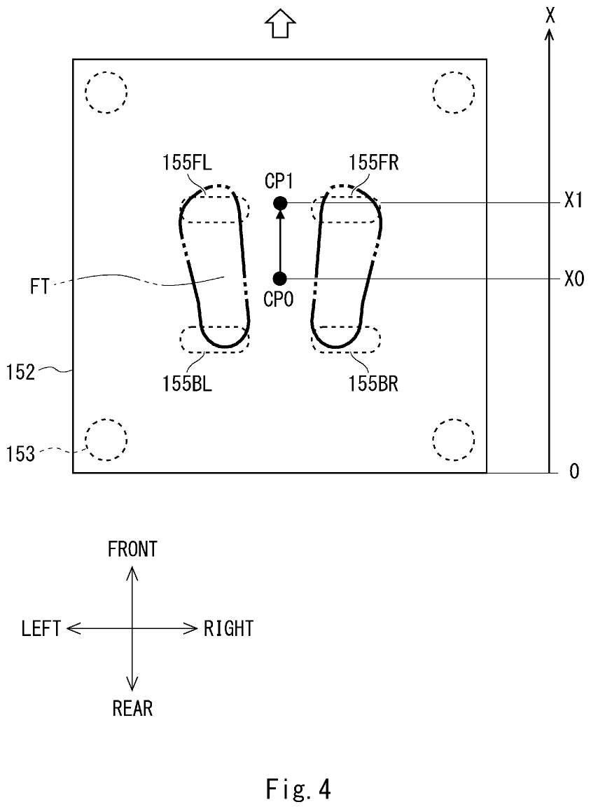

Next, an operation of the balance training system 100 will be described with reference to FIGS. 3 and 4. FIGS. 3 and 4 are diagrams for explaining the operation of the balance training system 100.

Note that, in the examples of FIGS. 3 and 4, the vibrator 155 is composed of four vibrators 155FR, 155FL, 155BR, and 155BL. The vibrator 155FR is disposed in a front part (a toe part) of the sole of the right foot of the trainee 900, the vibrator 155FL is disposed in a front part (a toe part) of the sole of the left foot of the trainee 900, the vibrator 155BR is disposed in a rear part (a heel part) of the sole of the right foot of the trainee 900, and the vibrator 155BL is disposed in a rear part (a heel part) of the sole of the left foot of the trainee 900.

Before the training is started, the trainee 900 brings his/her sole to a specified position in a central part of the belt 151 and thus his/her state becomes a stationary standing state. When the training is started, the trainee 900 performs training to maintain his/her balance by attempting to move his/her center of gravity without moving the sole from the position where the sole is brought into contact with the belt 151.

The control unit 160 calculates a position CP0 of the center of gravity of the trainee 900 in a stationary standing state before the training is started. Specifically, the control unit 160 calculates the initial position CP0 of the center of gravity of the trainee 900 based on the loads received from the right and left feet FT of the trainee 900, the loads being detected by the load sensors 153 disposed at the four corners of the rectangular riding plate 152. At this time, the control unit 160 also specifies an expected position CPx of the center of gravity as a moving destination of the center of gravity in accordance with a training level of the trainee 900.

When the training is started, the control unit 160 periodically calculates a position CP1 of the center of gravity of the trainee 900 during the balance training. In the example of FIG. 3, the trainee 900 is maintaining his/her posture in a stationary standing state during the balance training. Therefore, the position CP1 of the center of gravity of the trainee 900 is unchanged from the initial position CP0 of the center of gravity.

At this time, the control unit 160 vibrates the vibrator 155 so as to prompt the trainee 900 to move the position CP1 of his/her center of gravity from the initial position CP0 of his/her center of gravity to the expected position CPx thereof. In the example of FIG. 3, the position CPx of the center of gravity of the trainee 900 is located in front of the position CP0 of the center of gravity. Therefore, the control unit 160 vibrates the vibrators 155FR and 155FL (i.e., vibrations are applied to the toe parts of both feet of the trainee 900). By doing so, the trainee 900 can know that he/she has been instructed to move the position CP1 of his/her center of gravity forward. That is, the trainee 900 can perform the balance training while receiving support by the vibration of the vibrator.

Note that, when it is desired to give an instruction to move the position CP1 of the center of gravity of the trainee 900 diagonally forward to the right, the control unit 160 vibrates the vibrator 155FR (i.e., a vibration is applied to the toe of the right foot of the trainee 900). Further, when it is desired to give an instruction to move the position CP1 of the center of gravity of the trainee 900 diagonally forward to the left, the control unit 160 vibrates the vibrator 155FL (i.e., a vibration is applied to the toe of the left foot of the trainee 900). Further, when it is desired to give an instruction to move the position CP1 of the center of gravity of the trainee 900 diagonally backward to the right, the control unit 160 vibrates the vibrator 155BR (i.e., a vibration is applied to the heel of the right foot of the trainee 900). Further, when it is desired to give an instruction to move the position CP1 of the center of gravity of the trainee 900 diagonally backward to the left, the control unit 160 vibrates the vibrator 155BL (i.e., a vibration is applied to the heel of the left foot of the trainee 900). The above configuration enables the trainee 900 to know in which direction he/she should move his/her center of gravity based on the body part to which the vibration of the vibrator 155 has been applied.

Further, the control unit 160 increases the vibration of the vibrator 155 (in this example, the vibrators 155FR and 155FL) as a difference between the current position CP1 of the center of gravity of the trainee 900 and the expected position CPx thereof becomes larger. This configuration enables the trainee 900 to know how much he/she should move his/her center of gravity based on the magnitude of the vibration of the vibrator 155.

Note that, in this example, the control unit 160 vibrates the vibrators 155FR and 155FL when it gives an instruction to move the position CP1 of the center of gravity of the trainee 900 forward, but the present disclosure is not limited thereto. The control unit 160 may vibrate, for example, a vibrator (in this example, the vibrators 155BR and 155BL) located in a direction in which the center of gravity of the trainee 900 is not desired to be moved.

When the trainee 900 moves the position CP1 of his/her center of gravity to the position CPx of his/her center of gravity as instructed, the control unit 160 rotates the belt 151 in accordance with the movement vector (indicated by a solid arrow in FIG. 4; in this example, the movement vector from the position CP0 of the center of gravity of the trainee 900 to the position CPx of the center of gravity) of the position CP1 of the center of gravity of the trainee 900. The trainee 900 standing on the belt 151 also moves with the rotation of the belt 151. In this example, the belt 151 can rotate only in the front-rear direction.

The X-axis shown in FIG. 4 indicates the position of the center of gravity of the trainee 900 in the front-rear direction when the rear end of the riding plate 152 is defined as a starting point. In the example of FIG. 4, the position CP0 of the center of gravity of the trainee 900 in the X-axis direction is a position X0, and the position CP1 of the center of gravity in the X-axis direction is a position X1. The control unit 160 rotates the belt 151 forward or backward according to the difference between the positions X1 and X0. In the example of FIG. 4, the control unit 160 rotates the belt 151 forward according to the difference between the positions X1 and X0.

As described above, the balance training system 100 enables the trainee 900 to receive support by a vibration of the vibrator 155 even when he/she has difficulty in receiving voice support from an assistant due to hearing loss, higher brain disorder, or the like. Therefore, it is possible for the trainee 900 to perform effective balance training. (First modified example of balance training system 100)

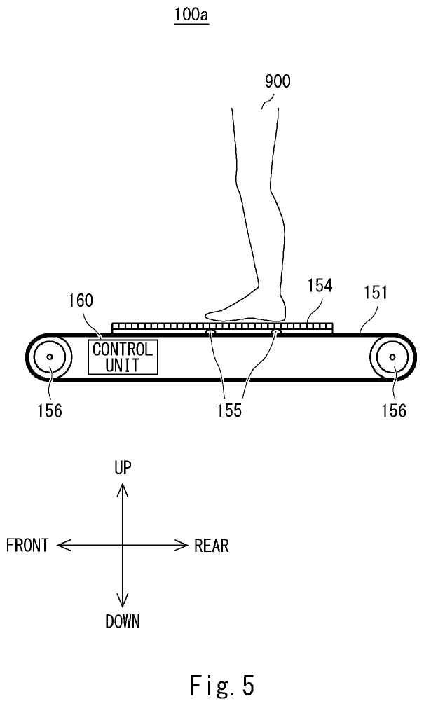

FIG. 5 is a schematic side view showing a first modified example of the balance training system 100 as a balance training system 100a.

In the balance training system 100a, the load detection part is not composed of the riding plate 152 and the load sensor 153, and is instead composed of a load distribution sensor 154. The details thereof will be described below.

The load distribution sensor 154 is composed of a plurality of sensors. The plurality of sensors are arranged in a matrix on a mounting surface for supporting the sole of the trainee 900 in a standing state. The load distribution sensor 154 can detect the distribution of the surface pressure received from the trainee 900's feet using the plurality of sensors. Therefore, as a matter of course, the load distribution sensor 154 can also detect loads received from the trainee 900.

The vibrator 155 is attached to the load distribution sensor 154, for example, at a predetermined position under the load distribution sensor 154 so that a vibration is transmitted to the sole of the foot of the trainee 900 standing on the mounting surface of the load distribution sensor 154.

The control unit 160 vibrates the vibrator 155 so as to prompt the trainee 900 to move the position of his/her center of gravity from the current position of his/her center of gravity to the expected position thereof. By doing so, the trainee 900 can perform training in which his/her center of gravity is moved to the expected position thereof while receiving support from the sole of the foot by the vibration of the vibrator 155.

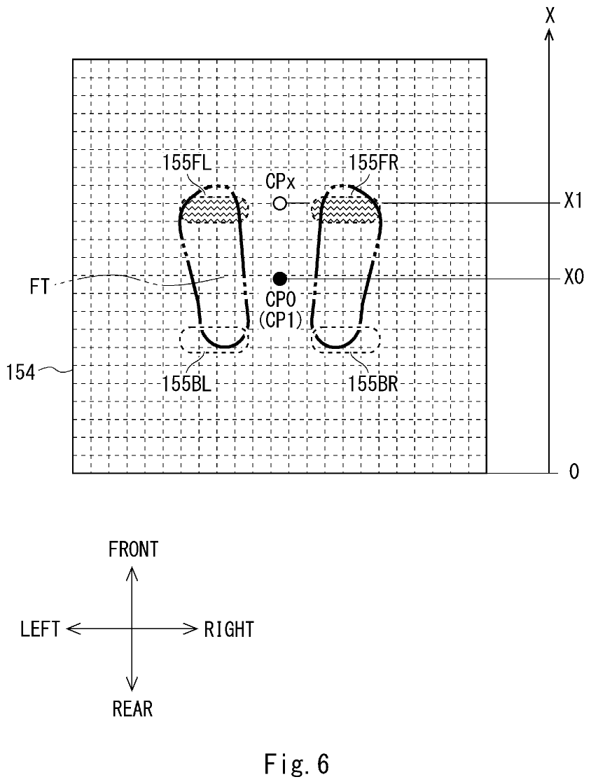

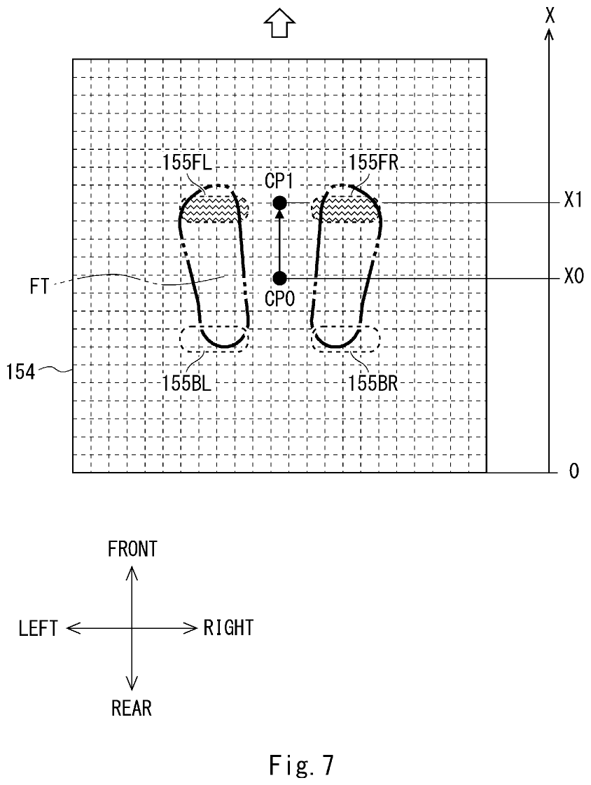

FIGS. 6 and 7 are diagrams for explaining the operation of the balance training system 100a. FIGS. 6 and 7 correspond to FIGS. 3 and 4, respectively. The configurations and operations of the balance training system 100a other than the above ones are similar to those of the balance training system 100, and thus the descriptions thereof will be omitted.

As described above, the balance training system 100a can provide advantageous effects equivalent to those of the balance training system 100. (Second modified example of balance training system 100)

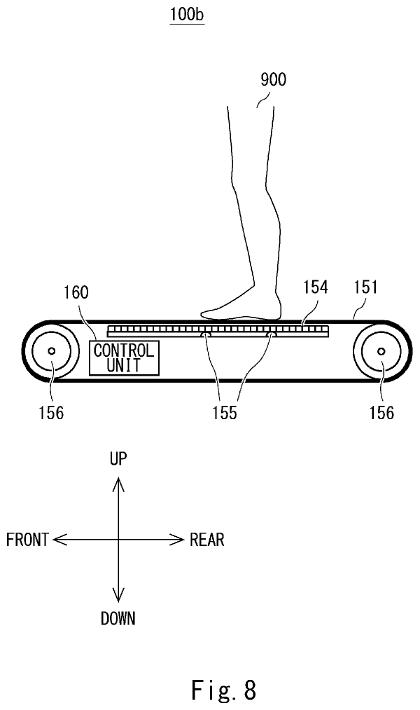

FIG. 8 is a schematic side view showing a second modified example of the balance training system 100 as a balance training system 100b.

In the balance training system 100b, unlike the balance training system 100a, the load distribution sensor 154 is disposed on an inner side of the ring-shaped belt 151 (i.e., under the surface of the belt 151 on which the trainee 900 rides (stands)).

The vibrator 155 is attached to the riding plate 152, for example, at a predetermined position under the riding plate 152 so that a vibration is transmitted to the sole of the foot of the trainee 900 standing on the riding plate 152 via the belt 151.

The control unit 160 vibrates the vibrator 155 so as to prompt the trainee 900 to move his/her position of the center of gravity from the current position of his/her center of gravity to the expected position thereof. By doing so, the trainee 900 can perform training in which his/her center of gravity is moved to the expected position thereof while receiving support from the sole of the foot by the vibration of the vibrator 155.

The configurations and operations of the balance training system 100b other than the above ones are similar to those of the balance training system 100, and thus the descriptions thereof will be omitted.

As described above, the balance training system 100b can provide advantageous effects equivalent to those of the balance training system 100.

### Second Embodiment

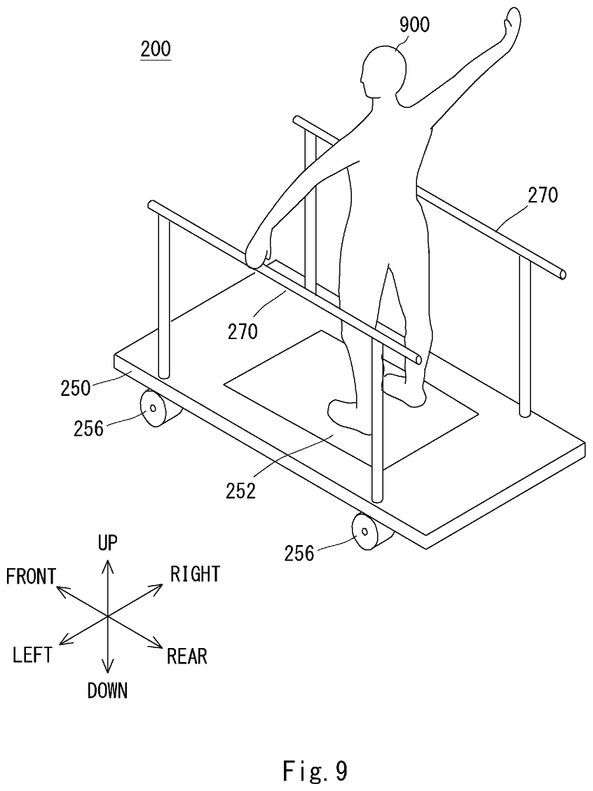

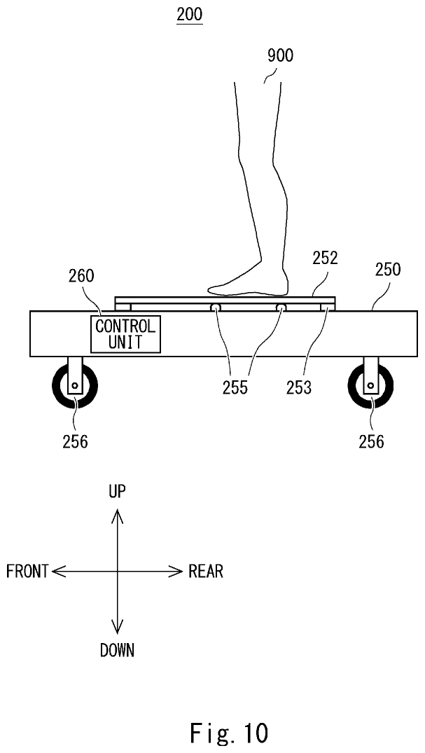

FIG. 9 is a schematic perspective view (viewed from diagonally backward to the left) of a balance training system 200 according to a second embodiment. FIG. 10 is a schematic side view (viewed from the left) of a part of the balance training system 200. The balance training system 200 may also be referred to as a balance training apparatus.

The balance training system 200 includes a moving carriage (a mobile body) 250, a riding plate 252, a load sensor 253, a vibrator 255, a control unit 260, and a handrail 270. The riding plate 252 and the load sensor 253 comprise the load detection part. The riding plate 252, the load sensor 253, the vibrator 255, the control unit 260, and the handrail 270 correspond to the riding plate 152, the load sensor 153, the vibrator 155, the control unit 160, and the handrail 170, respectively. Note that, in the following description, the up-down direction, the right-left direction, and the front-rear direction are directions based on the orientation of the trainee 900.

The moving carriage 250 is configured to be movable in the front-rear direction on a moving surface of a floor surface or the like of a rehabilitation facility as the moving surface. Further, the load detection part composed of the load sensor 253 and the riding plate 252 and the vibrator 255 are mounted on the moving carriage 250.

The riding plate 252 is a riding part on which the trainee 900 rides, and has a mounting surface for supporting the sole of the trainee 900 in a standing state. As the riding plate 252, a rectangular flat plate made of, for example, polycarbonate resin, which has relatively high rigidity to withstand the riding of the trainee 900, is used. The riding plate 252 is supported on an upper surface of the moving carriage 250 with the load sensors 253 arranged at four corners interposed therebetween.

The load sensor 253 is, for example, a load cell, and detects a load received from the foot of the trainee 900 standing on the riding plate 252. The load sensors 253 are arranged at four corners of the riding plate 252 and support the riding plate 252.

The handrail 270 is provided so as to be positioned, for example, on the side of the trainee 900 so that it can be grasped when he/she is about to lose his/her balance or when he/she feels uneasy.

The control unit 260 calculates the position of the center of gravity of the trainee 900 from the load detected by the load sensor 253 and rotates wheels 256 at a speed, in a direction, and by an amount according to a movement vector (a moving direction and an amount of movement) of the calculated position of the center of gravity of the trainee 900, thereby moving the moving carriage 250. The trainee 900 standing on the moving carriage 250 also moves with the movement of the moving carriage 250.

The control unit 260 also controls a vibration of the vibrator 255. The vibrator 255 is attached to the riding plate 252, for example, at a predetermined position under the riding plate 252 so that a vibration is transmitted to the sole of the foot of the trainee 900 standing on the riding plate 252. As the vibrator 255 is provided under the riding plate 252, the load detection part can prevent an erroneous detection due to the load of the vibrator 255, unlike in the case in which the vibrator 255 is mounted on the riding plate 252.

The control unit 260 vibrates the vibrator 255 so as to prompt the trainee 900 to move the position of his/her center of gravity from the current position of his/her center of gravity to the expected position thereof (i.e., based on the movement vector from the current position of the center of gravity to the expected position thereof). By doing so, the trainee 900 can perform training in which his/her center of gravity is moved to the expected position thereof while receiving support from the sole of the foot by the vibration of the vibrator 255.

As described above, the balance training system 200 enables the trainee 900 to receive support by a vibration of the vibrator 255 even when he/she has difficulty in receiving voice support from an assistant due to hearing loss, higher brain disorder, or the like. Therefore, it is possible for the trainee 900 to perform effective balance training.

### (Modified Example of Balance Training System 200)

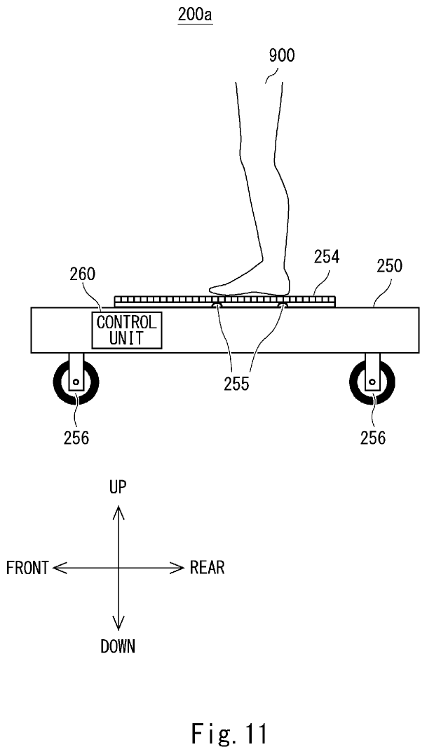

FIG. 11 is a schematic side view showing a modified example of the balance training system 200 as a balance training system 200a.

In the balance training system 200a, the load detection part is not composed of the riding plate 252 and the load sensor 253, and is instead composed of a load distribution sensor 254. The details thereof will be described below.

The load distribution sensor 254 is composed of a plurality of sensors. The plurality of sensors are arranged in a matrix on a mounting surface for supporting the sole of the trainee 900 in a standing state. The load distribution sensor 254 can detect the distribution of the surface pressure received from the trainee 900's feet using the plurality of sensors. Therefore, as a matter of course, the load distribution sensor 254 can also detect loads received from the trainee 900.

The vibrator 255 is attached to the load distribution sensor 254, for example, at a predetermined position under the load distribution sensor 254 so that a vibration is transmitted to the sole of the foot of the trainee 900 standing on the mounting surface of the load distribution sensor 254.

The control unit 260 vibrates the vibrator 255 so as to prompt the trainee 900 to move the position of his/her center of gravity from the current position of his/her center of gravity to the expected position thereof. By doing so, the trainee 900 can perform training in which his/her center of gravity is moved to the expected position thereof while receiving support from the sole of the foot by the vibration of the vibrator 255.

The configurations and operations of the balance training system 200a other than the above ones are similar to those of the balance training system 200, and thus the descriptions thereof will be omitted.

As described above, the balance training system 200a can provide advantageous effects equivalent to those of the balance training system 200.

The present disclosure is not limited to the first and second embodiments described above, and may be modified as appropriate without departing from the spirit of the disclosure.

In the first embodiment, an example in which the vibrator 155 is provided under the load detection part has been described. However, the present disclosure is not limited to this. The vibrator 155 may be attached, for example, to a predetermined body part (e.g., toe and heel parts of each foot) of the trainee 900. By doing so, the trainee 900 can receive the vibration of the vibrator 155 more properly.

Similarly, in the second embodiment, although an example in which the vibrator 255 is provided under the load detection part has been described, the present disclosure is not limited to this. The vibrator 255 may be attached, for example, to a predetermined body part (e.g., toe and heel parts of each foot) of the trainee 900. By doing so, the trainee 900 can receive the vibration of the vibrator 255 more properly.

Further, in the first embodiment, an example in which the control unit 160 rotates the belt 151 in the front-rear direction in accordance with the movement vector of the position CP1 of the center of gravity of the trainee 900 has been described. However, the present disclosure is not limited to this. If the belt 151 is configured to be rotatable not only in the front-rear direction but also in the right-left direction, the control unit 160 can rotate the belt 151 in the front-rear and right-left directions in accordance with the movement vector of the position CP1 of the center of gravity of the trainee 900.

Similarly, in the second embodiment, an example in which the control unit 260 moves the moving carriage 250 in the front-rear direction in accordance with the movement vector of the position CP1 of the center of gravity of the trainee 900 has been described. However, the present disclosure is not limited to this. If the moving carriage 250 is configured to be movable not only in the front-rear direction but also in the right-left direction, the control unit 260 can move the moving carriage 250 in the front-rear and right-left directions in accordance with the movement vector of the position CP1 of the center of gravity of the trainee 900.

In the first embodiment, an example in which the control unit 160 is included in the treadmill 150 has been described. However, the present disclosure is not limited to this. The control unit 160 may be provided outside the treadmill 150, or may be configured to remotely control the treadmill 150. Similarly, in second embodiment, although an in which the control unit 260 is included in the moving carriage 250 has been explained, the present disclosure is not limited to this. The control unit 260 may be provided outside the moving carriage 250, or may be configured to remotely control the moving carriage 250.

Further, although the present disclosure has been described in the above embodiments as a hardware configuration, the present disclosure is not limited to this. The present disclosure can be realized by causing a CPU (Central Processing Unit) to execute a computer program for controlling a balance training system.

Further, the above-described program can be stored and provided to a computer using any type of non-transitory computer readable media. Non-transitory computer readable media include any type of tangible storage media.

Examples of non-transitory computer readable media include magnetic storage media (such as floppy disks, magnetic tapes, hard disk drives, etc.), optical magnetic storage media (e.g., magneto-optical disks), CD-ROM (compact disc read only memory), CD-R (compact disc recordable), CD-R/W (compact disc rewritable), and semiconductor memories (such as mask ROM, PROM (programmable ROM), EPROM (erasable PROM), flash ROM, RAM (random access memory), etc.). The program may be provided to a computer using any type of transitory computer readable media. Examples of transitory computer readable media include electric signals, optical signals, and electromagnetic waves. Transitory computer readable media can provide the program to a computer via a wired communication line (e.g., electric wires, and optical fibers) or a wireless communication line.

From the disclosure thus described, it will be obvious that the embodiments of the disclosure may be varied in many ways. Such variations are not to be regarded as a departure from the spirit and scope of the disclosure, and all such modifications as would be obvious to one skilled in the art are intended for inclusion within the scope of the following claims.

What is claimed is:

1. A balance training system comprising:
a load detection part including a mounting surface for supporting soles of feet of a trainee in a standing state, the load detection part being configured to detect a load received from the trainee riding on the mounting surface;
a mobile body, the load detection part being attached to the mobile body;
a control unit configured to calculate a position of a center of gravity of the trainee based on the load detected by the load detection part and control a movement of the mobile body based on a change of the position of the center of gravity of the trainee; and
a vibrator that is provided so as to be able to apply a vibration to the trainee,
wherein the control unit is configured to vibrate the vibrator so as to prompt the trainee to move the position of the center of gravity of the trainee from a current position of the center of gravity to an expected position thereof.

2. The balance training system according to claim 1, wherein the control unit is configured to increase the vibration of the vibrator as a difference between the current position of the center of gravity and the expected position thereof becomes larger.

3. The balance training system according to claim 1, wherein the control unit is configured to apply the vibration of the vibrator to a body part of the trainee according to a direction from the current position of the center of gravity to the expected position thereof.

4. The balance training system according to claim 3, wherein the control unit is configured to apply the vibration of the vibrator to one of a toe and a heel of the foot of the trainee determined in accordance with the direction from the current position of the center of gravity to the expected position thereof.

5. The balance training system according to claim 1, wherein the vibrator is provided under the load detection part.

6. The balance training system according to claim 1, wherein the vibrator is attached to a predetermined body part of the trainee.

7. A method of controlling a balance training system, the method comprising:
detecting, by using a load detection part including a mounting surface for supporting soles of feet of a trainee in a standing state, a load received from the trainee riding on the mounting surface; and
calculating a position of a center of gravity of the trainee based on the load detected by the load detection part and controlling a movement of a mobile body, to which the load detection part is attached, based on a change of the position of the center of gravity of the trainee,
wherein in the controlling of the movement of the mobile body, a vibrator is vibrated so as to prompt the trainee to move the position of the center of gravity of the trainee from a current position of the center of gravity to an expected position thereof.

8. A non-transitory computer readable medium storing a control program for causing a computer to:
detect, by using a load detection part including a mounting surface for supporting soles of feet of a trainee in a standing state, a load received from the trainee riding on the mounting surface; and
calculate a position of a center of gravity of the trainee based on the load detected by the load detection part and control a movement of a mobile body, to which the load detection part is attached, based on a change of the position of the center of gravity of the trainee,
wherein in the controlling of the movement of the mobile body, a vibrator is vibrated so as to prompt the trainee to move the position of the center of gravity of the trainee from a current position of the center of gravity to an expected position thereof.
